(19) United States
(12) Patent Application Publication
(10) Pub. No.: US 2009/0105725 A1
(43) Pub. Date: Apr. 23, 2009

(54) STEREOTACTIC HEAD FRAME LOCALIZER

(75) Inventors: James P. O'Connor, Billerica, MA (US); Russell Copley, Dracut, MA (US); William Keller, Chelmsford, MA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: INTEGRA RADIONICS, INC., PLAINSBORO, NJ (US)

(21) Appl. No.: 11/720,431

(22) PCT Filed: Sep. 27, 2005
(86) PCT No.: PCT/US05/34824

§ 371 Date: Nov. 5, 2007

(51) Int. Cl. A61B 19/00

(52) U.S. Cl. 606/130

(57) ABSTRACT

A stereotactic head frame localizer having a plurality of fiducial rods disposed in a plurality of sides is disclosed. The stereotactic head frame localizer includes five connected sides with each side including a plurality of the fiducial rods. One of the sides may be removably attached to the stereotactic head frame localizer. The fiducial rods include a material or may be formed from a material that, in cooperation with a selected imaging modality (i.e. CT, MRI, or PET), will appear on the resulting image scan as image points. The fiducial rods may be disposed in an orientation that is horizontal, vertical, or diagonal to an axis of a body of a patient. The stereotactic head frame localizer may be used in cooperation with a head ring attached to a patient for determining the location of a target in an x, y, and z coordinate system.

### CROSS REFERENCE TO RELATED APPLICATIONS

This application claims priority from U.S. Provisional Application Ser. No. 60/614,608, filed on Sep. 30, 2004, the contents of which are hereby incorporated by reference in their entirety.

### BACKGROUND

1. Field of the Invention

The present disclosure relates to a surgical apparatus. More particularly, the present disclosure relates to a stereotactic head frame localizer and associated fiducial rods for use in stereotactic surgery.

2. Background of the Art

The use of computed tomography (CT) imaging methods in medicine is widespread. It is commonplace to attach frames or mechanical devices to the patient during CT scanning. A common application is in brain surgery where a head ring is attached to the patient's skull for the purpose of providing a reference platform at the time of CT image scanning. Typically, the head ring is fastened directly to the patient's skull by head posts and skull fixation structures, such as sharpened, pointed screws on the head posts that anchor directly to the skull. The head ring may then be used as a rigid platform onto which a localizer structure may be attached. When a patient is scanned with the localizer structure rigidly attached to his or her skull, index marks from the localizer structure will appear on the scan slice images and provide data for mathematically determining the coordinates of every image point seen in the CT slice relative to the localizer structure. This is described in detail in U.S. Pat. No. 4,608,977 to Brown, the contents of which is hereby incorporated by reference in its entirety. Conventional localizer systems have enabled image points to be determined only for axial slices, which are the type that the CT scan provides. Magnetic Resonance Imaging (MRI) and Positron Emission Tomography (PET) scanners enable scan slices in nearly any arbitrary plane, including the sagittal and coronal planes. The present disclosure is related to determining target coordinates from various plane slices including axial slices, sagittal slices, and coronal slices.

In conventional systems, it is difficult to determine the x, y, and z coordinates of a target if the scan plane were substantially in the sagittal or coronal orientations such that the axial rods and their respective diagonals were not cut by the scan plane. In conventional systems, the localizer structure may include fluid filled portions. The fluid is generally opaque to the imaging modality and interacts with the imaging modality to cause index marks to appear on the scan slices. In these systems, fluid loss due to evaporation or other reasons results in degraded system performance and possibly a loss of image scan data.

Thus, it is an object of the present disclosure to provide a stereotactic head frame localizer which can determine target coordinates for axial scan cuts and also for scan cuts which are substantially parallel to the sagittal or coronal planes, or, moreover, for any planar scan cut through the subject.

Another object of the present disclosure is to provide a stereotactic head frame localizer which minimizes losses of fluids or other materials that interact with the selected imaging modality.

A further object of the present disclosure is to provide fiducial rods which are permanently sealed against opening by the user.

### SUMMARY

The present disclosure is directed towards a stereotactic head frame localizer including a plurality of connected sides and a plurality of fiducial rods. The stereotactic head frame localizer includes an anterior side, a left side, a right side, and a top side that are connected to each other. A posterior side may be removably attached to the stereotactic head frame localizer in a location opposite the anterior side. The posterior side may include a number of throughholes and attachment members where the throughholes and attachment members are configured and adapted for cooperation with a corresponding number of respective posts and bores disposed on the stereotactic head frame localizer. Each of the sides includes a plurality of fiducial rods. A head ring may be attached to the head of a patient. The stereotactic head frame localizer may be attached to the head ring by using a plurality of fixation devices.

Each fiducial rod is a sealed elongate tube including a selected indicator. The fiducial rods may be permanently sealed against opening by the user. The sealed fiducial rod minimizes evaporative or other losses of the indicator. Alternatively, each fiducial rod may be formed from a material that is selected to have desirable imaging properties for the imaging modality in use (i.e. CT, MRI, or PET). The fiducial rods are arranged in the stereotactic head frame localizer such that a scan along the axial, sagittal, or coronal planes produces image points corresponding to the fiducial rods. These image points are useful in determining the location of a target in an x, y, and z coordinate system in relation to the stereotactic head frame localizer.

### BRIEF DESCRIPTION OF THE DRAWINGS

Embodiments of the presently disclosed stereotactic head frame localizer are described hereinbelow with reference to the drawings wherein:

FIG. 1 is a perspective view of the axial, coronal, and sagittal planes and associated x, y, z coordinate axes relative to a body;

FIG. 2A is a perspective view of a conventional localizer system having a head ring and an attached head frame, the localizer system being positioned about a head of a patient;

FIG. 2B is a perspective view of another conventional head frame localizer for use with the localizer system of FIG. 2A;

FIG. 3A illustrates tomographic images using the localizer system of FIG. 2A;

FIG. 3B illustrates tomographic images using the localizer system of FIG. 2B;

FIG. 4A is a perspective view of another localizer system having a stereotactic head frame localizer according to an embodiment of the present disclosure;

FIG. 4B is a front view of the stereotactic head frame localizer of FIG. 4A;

FIG. 4C is a rear view of the stereotactic head frame localizer of FIG. 4A;

FIG. 4D is a side view of the stereotactic head frame localizer of FIG. 4A;

FIG. 4E is a top view of the stereotactic head frame localizer of FIG. 4A;

FIG. 4F is a perspective view of a fiducial rod;

FIGS. 5 A-C are perspective views of the stereotactic head frame localizer of FIG. 4 illustrating the axial, sagittal, and coronal tomographic planes intersecting the stereotactic head frame localizer; and

FIGS. 6A-C illustrate the tomographic images of the planes shown in FIGS. 5A-C respectively.

### DETAILED DESCRIPTION

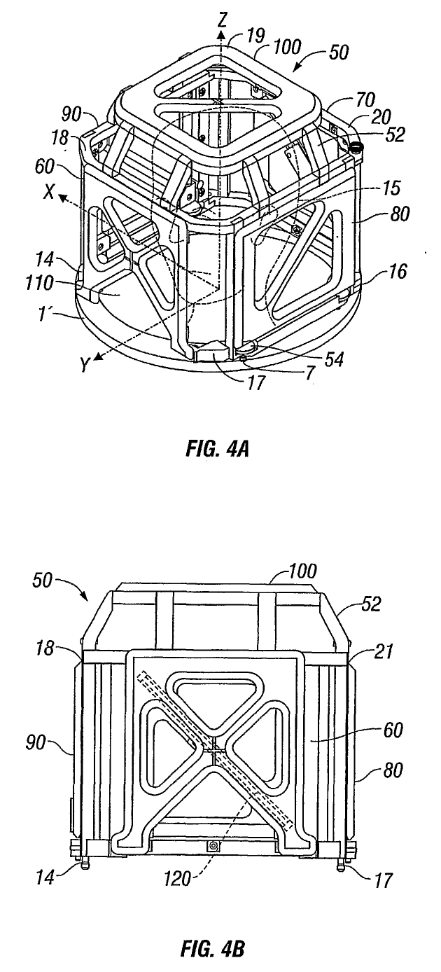

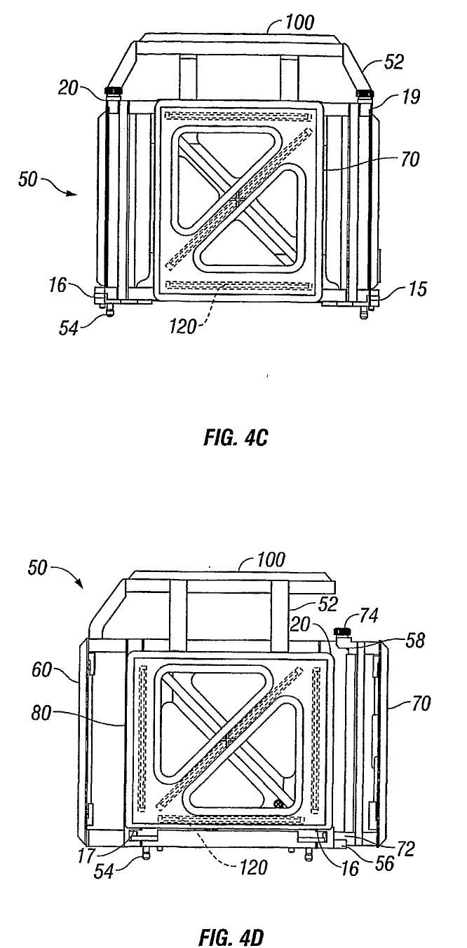

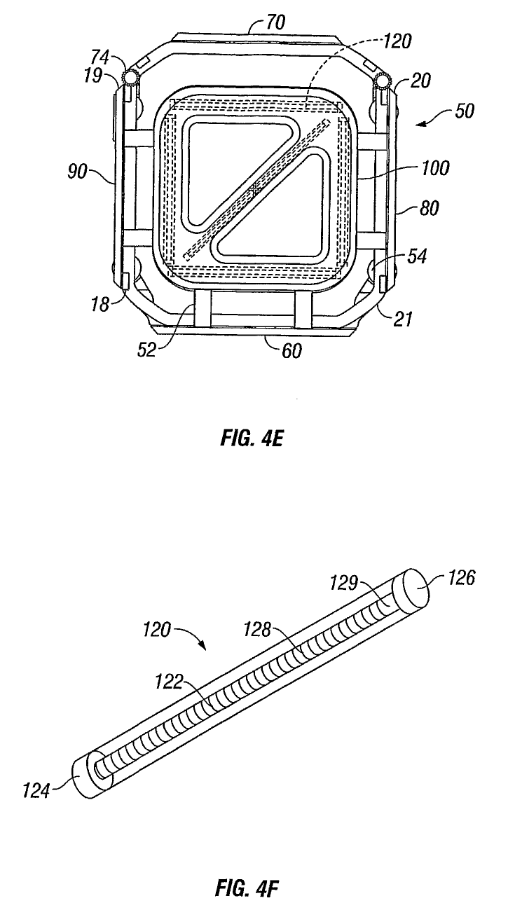

Embodiments of the presently disclosed stereotactic head frame localizer will now be described in detail with reference to the drawings, in which like reference numerals designate identical or corresponding elements in each of the several views. The presently disclosed stereotactic head frame localizer 50, or localizer system is shown in FIG. 4A and described in detail hereinafter, is a device for determining a precise location of a target in a coordinate system (i.e. localizing a target) in relation to stereotactic head frame localizer 50 which is attached to a head ring on a body being scanned using a tomographic scan (i.e. CT, MRI, or PET), where the scan plane or scan cut is in virtually any orientation. This includes axial planes as well as planes that are more nearly parallel to the sagittal or coronal planes. The presently disclosed stereotactic head frame localizer enables selecting frame-related coordinates for targets seen in any slice or plane through the subject, parallel to these ideal axial, coronal, or sagittal planes or not.

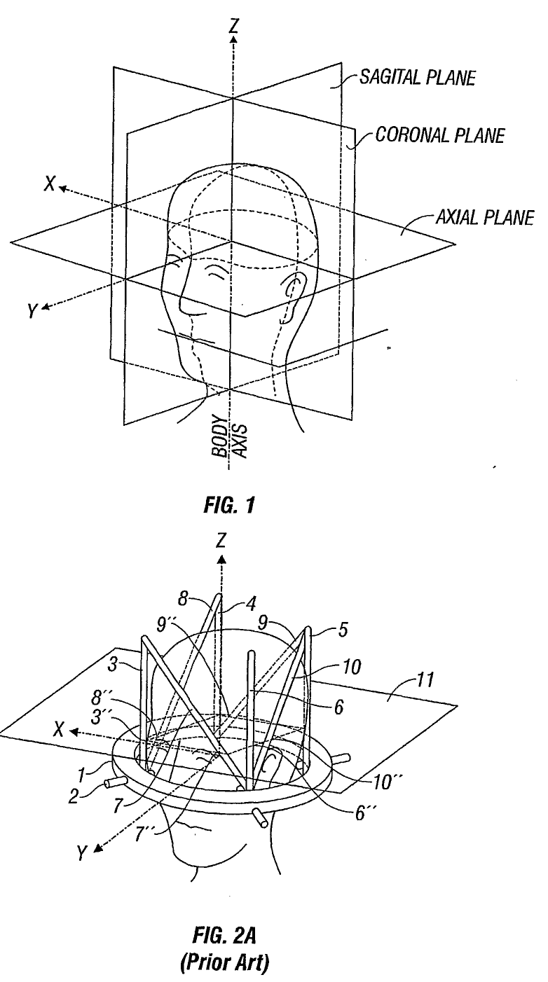

FIG. 1 shows a head of a patient and a superposed with x, y, z coordinate system. The body axis is defined by the Z-axis, and one planar slice perpendicular to the Z-axis will be referred to as an axial slice or plane which extends along the Y-axis. Since there is no precise body axis, these terms refer to axes and planes that are substantially parallel and/or perpendicular to the body axis, or Z-axis, respectively. They may be precisely defined relative to stereotactic head frame localizer 50 that is affixed to the body where the axial axis of stereotactic head frame localizer 50 is substantially parallel to the body axis. The x and y coordinate axes are defined to be perpendicular to the sagittal and coronal planes, respectively, as shown in FIG. 1. In the CT scan images, axial plane slices are standard. Presently, scanners (i.e. MRI or PET) are capable of obtaining scan slices in the sagittal, coronal, or other planes as desired. The sagittal plane is parallel to a plane roughly going through the mid-line of the head (i.e. nose and midway between the ears) while the coronal plane is parallel to a plane that goes through the ears and up over the crown of the head.

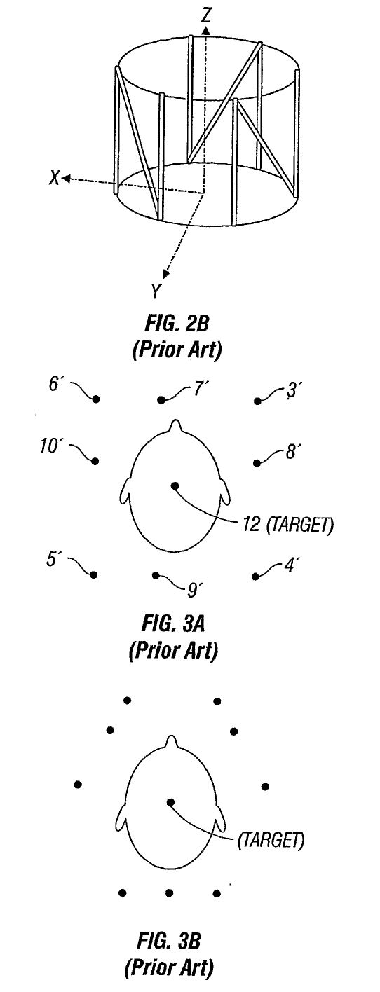

A conventional localizer for determining coordinates in axial scans is illustrated in FIG. 2A and fully described in U.S. Pat. No. 4,618,978 to Cosman, the contents of which are hereby incorporated by reference in their entirety. A head ring 1 is attached to a skull by a plurality of screws 2. This provides a rigid apparatus in a fixed position relative to the body. Fastened to head ring 1 is a rod system, with vertical rods 3, 4, 5, and 6 being essentially parallel to the body axis (i.e. the Z-axis). A plurality of diagonal rods 7, 8, 9, and 10 are also included and interposed with the vertical rods 3, 4, 5, and 6 as illustrated in FIGS. 1 and 2A. When an essentially axial scan cut 11 (i.e. along the axial plane) is taken through the head, then vertical rods 3, 4, 5, and 6, and diagonal rods 7, 8, 9, and 10 appear as localizer image spots on the CT scan image. These image spots correspond to points 3′, 4′, 5′, 6′ and 7′, 8′, 9′, 10′, respectively, as shown in FIG. 3A. In addition, one might identify a target spot 12 on the CT image of FIG. 3A. By knowing the proportional distances of the diagonal points from their respective adjacent rods in the image of FIG. 3A, and knowing their actual physical positions on the localizer of FIG. 2A, it is possible to calculate the x, y, and z positions of each of the diagonal intersection points 7″, 8″, 9″, and 10″ of the diagonal rods and the scan plane as shown in FIG. 2A. This information enables determination of the equation of plane 11 relative to the x, y, and z coordinate system which is related to head ring 1. The x, y, and z coordinates of the target relative to the head ring 1 may be determined from the position of target image 12 in the CT image using a selected calculation, such as a proportional vector calculation. The x, y, and z coordinates of at least three of diagonal intersections 7″, 8″, 9″, and 10″ are necessary to determine plane 11, so that the fourth diagonal intersection may be used as a check or for greater accuracy. If head ring 1 is clamped parallel to scan plane 11, only one diagonal intersection of the four shown is required to determine the z position of plane 11 and, thus, the equation of plane 11.

Once the x, y, and z coordinates of the target are known relative to head ring 1, then a stereotaxic guidance system can be attached to head ring 1 and an instrument directed for precisely reaching the target. This is one of the uses of the localizer system i.e., as part of a stereotaxic guide. Examples of such systems are disclosed in U.S. Pat. No. 6,662,036 to Cosman and U.S. Pat. No. 6,675,040 to Cosman, the contents of each being hereby incorporated by reference in their entirety.

FIG. 2B shows another conventional localizer system, the BRW Brown-Roberts-Wells localizer system. It includes six vertical or axial rods and three diagonal rods for determining the three diagonal-plane intersection coordinates, and thus the scan plane, even if the scan plane is not parallel to the frame plane. FIG. 3B shows a corresponding scan plane image with vertical rod points, diagonal rod points, and target image points.

Referring to FIG. 4A, an embodiment of the presently disclosed stereotactic head frame localizer is shown generally as 50 and is adapted for scanning through the head. The subject's head is shown in phantom (i.e. dashed lines). Stereotactic head frame localizer 50 is a substantially open structure including an anterior or front side 60, a posterior, or rear side 70, and adjoining left and right sides 80, 90. A plurality of frame members 52 connect a top side 100 to anterior side 60, left side 80, and right side 90 as described hereinafter. One end of each of frame members 52 is attached to anterior side 60, left side 70, and right side 80. An opposing end of each of frame members 52 is attached to a surface of top side 100 for fixedly attaching top side 100 to stereotactic head frame localizer 50. In one embodiment, frame members 52 extend substantially vertically from their respective sides and angle inwards towards the Z-axis. Each side 60, 70, 80, 90, and 100 may be formed from a material that is substantially transparent to the selected imaging modality.

An opening 110 is defined by anterior side 60, posterior side 70, left side 80, and right side 90 where opening 110 is opposite top side 100. In addition, opening 110 is configured and dimensioned to accommodate the head of a patient.

Posterior side 70 (FIG. 4C) includes a plurality of fiducial rods 120 (FIG. 4F), shown in phantom, and is releasably attached to stereotactic head frame localizer 50. A plurality of arbitrary points 14, 15, 16, 17, 18, 19, 20, and 21 are illustrated in FIGS. 4B-E and the orientation of each fiducial rod 120 is designated by the pair of numbers which specify its end points. In one embodiment, posterior side 70 includes at least one fiducial rod 120 disposed along a line defined between points 15-16, at least one fiducial rod 120 disposed along a line defined between points 16-19, and at least one fiducial rod 120 is disposed along a line defined between points 19-20. In another embodiment, fiducial rod 120 extending between points 15-16 or 19-20 is relatively larger than other fiducial rods 120 in posterior side 70 thereby producing a larger image spot in the resulting image scan. This larger image spot may be used by system algorithms to identify rod locations in stereotactic head frame localizer 50, the orientation of stereotactic head frame localizer 50 relative to head ring 1′, or for other identification purposes. Although at least one fiducial rod 120 is described as existing between points 16-19, it is contemplated that this fiducial rod 120 may alternately be disposed between points 15-20 without affecting the operability of stereotactic head frame localizer 50.

A pair of throughholes 72 is disposed on a bottom end of posterior side 70 where each throughhole 72 is adapted for sliding engagement with a corresponding pair of posts 56 of stereotactic head frame localizer 50 (see FIG. 4D). A pair of attaching members 74 is disposed on an opposed upper end of posterior side 70 and are each adapted for sliding engagement with a pair of bores 58 of stereotactic head frame localizer 50 (see FIG. 4D). In one embodiment, each bore 58 is threaded for engaging a complementary thread arrangement on each attaching member 74. In addition, each attaching member 74 may include a thumbwheel for the convenience of the operator.

By providing throughholes 72 and attaching members 74 that cooperate with respective posts 56 and bores 58, posterior side 70 may be securely attached to stereotactic head frame localizer 50 or readily removed from stereotactic head frame localizer 50 as desired. Removal of posterior side 70 is accomplished as follows. Attaching members 74 are withdrawn in a generally vertical direction from bores 58 either by disengaging their respective threads or by simple pulling. Once attaching members 74 are disengaged from bores 58, posterior side 70 may be raised in a generally vertical direction to separate throughholes 72 from their respective posts 56 thereby separating posterior side 70 from stereotactic head frame localizer 50. This arrangement allows access to regions of the patients head for inspection, surgical procedures, placement of fiducial markers, or other reasons related to stereotactic imaging.

Left side 80 and right side 90 are substantially similar structures (see FIG. 4D). Left side 80 includes at least one fiducial rod 120 disposed along a line defined between points 20-21 (FIG. 4E), at least one fiducial rod 120 disposed along a line defined between points 17-20 (FIG. 4D), and at least one fiducial rod 120 is disposed along a line defined between points 16-17. Alternately, fiducial rod 120 extending between points 17-20 may be replaced by fiducial rod 120 extending between points 16-21 without affecting the operability of stereotactic head frame localizer 50.

Right side 90 includes at least one fiducial rod 120 disposed along a line defined between points 18-19 (FIG. 4E), at least one fiducial rod 120 disposed along a line defined between points 14-19, and at least one fiducial rod 120 is disposed along a line defined between points 14-15. Similar to left side 80, fiducial rod 120 extending between points 14-19 may be replaced by fiducial rod 120 extending between points 15-18 without affecting operability of stereotactic head frame localizer 50.

Fiducial rods 120 that extend along a line defined by points 14-19, 17-18, 18-20, 16-19, 17-20 may alternately be referred to hereinafter as diagonals, diagonal rods, or diagonally oriented rods.

Left and right sides 80, 90 each include at least one fixation device 54 that is disposed on a bottom portion of each of the respective sides 80, 90 for attaching stereotactic head frame localizer 50 to head ring 1. In one embodiment, each fixation device 54 is threaded and includes a thumbwheel. By providing readily accessible fixation devices 54, stereotactic head frame localizer 50 is easily attached to and removed from head ring 1′ (FIG. 4A). In one embodiment, stereotactic head frame localizer 50 may be installed from above head ring 1′ using fixation devices 54 and corresponding orifices 7 in head ring 1′ (FIG. 4A).

With reference now to FIG. 4E, top side 100 includes a plurality of fiducial rods 120. Fiducial rods 120 are arranged such that one fiducial rod 120 is disposed on each of the four sides of top side 120. In other words, one fiducial rod 120 is disposed along each of the lines defined between points 18-19, 19-20, 20-21, and 18-21. In addition, another fiducial rod 120 is disposed along a diagonal line defined between points 18-20 or 19-21. Top side 100 is connected to stereotactic head frame localizer 50 by frame members 52 as previously discussed.

As seen in FIG. 4F, fiducial rod 120 is a generally an elongate tubular structure having a core 122 therein with opposed ends 124, 126. An indicator 128 having desirable imaging properties such that each fiducial rod 120 appears as an index mark on the resulting image scan is disposed in core 122. Each fiducial rod 120 may be partially filled with indicator 128 thereby providing a gap or space 129 in core 122. The size or volume of space 129 may be determined by operating characteristics such as the temperature range during storage, shipping, or operation in addition to the physical characteristics of indicator 128 and/or the physical characteristics of fiducial rod 120. In selecting the size or volume of space 129, damage to fiducial rod 120 and subsequent loss of indicator 128 is thereby minimized.

In one embodiment, indicator 128 is an MRI visible material such as would be known to a person of ordinary skill in the art. One example of such an indicator having the desired characteristics is a commercially available gel-like material supplied by Computerized Imaging Reference Systems, Inc. (CRS, Inc.) of Norfolk, Va. Alternately, indicator 128 may be a liquid, solid, or semi-solid material so long as it has the desired imaging properties for the selected imaging modality. Further still, each fiducial rod 128 may be formed from a selected material, as is known in the art, that has the desired imaging properties for the selected imaging modality (i.e. CT, MRI, or PET). For example, each fiducial rod 120 may be made of or include carbon fiber for CT scanning, be filled with a solution for MRI scanning, or be filled with one or more radioactive sources for PET scanning. In a particular embodiment, fiducial rods 120 may be made of glass such that the rods may be differentiated from the rest of the frame structure.

In a particular embodiment, unlike the prior art fiducial rods, rods 120 do not require or allow any filling by the user, and are permanently sealed vessels filled with a proprietary material, such as that which may be provided by CRS, Inc. Rods 120 are replaceable, and the device should be returned to the manufacture for annual service and recalibration.

One of opposed ends 124, 126 of each fiducial rod 120 is sealed prior to filling fiducial rod 120 with the desired indicator 128. After the desired quantity of indicator 128 is disposed in core 122, the other end 126, 124 is sealed thereby providing space 129, as previously discussed and enclosing indicator 128 inside fiducial rod 120. One end 124, 126 may be sealed during the manufacture of fiducial rod 120 where it is formed with only one open end. After filling, the remaining open end 126, 124 is sealed using techniques and structures as are known in the art. Examples of seals for fiducial rod 120 include plugs, caps, or other sealing structures as are known in the art. In addition, an adhesive may be used in cooperation with the selected sealing structure to improve the integrity of the seal thereby minimizing evaporative or other losses of indicator 128.

In another embodiment, fiducial rod 120 has a closed end and an open end. The open end is configured and dimensioned such that it has a large enough opening to permit the addition of an indicator, but the opening is of such a size that the open end can be heat sealed without causing evaporative losses of the indicator or damage to the fiducial rod. In this configuration, the fiducial rod may be formed from glass or another suitable material and the open end is a tapered opening thereby allowing the introduction of the indicator into the core of the fiducial rod and permitting rapid sealing of the open end by heat or other means without damaging the fiducial rod or losing any of the indicator.

By enclosing or sealing each fiducial rod 120, indicator 128 is retained within the confines of fiducial rod 120 thereby minimizing evaporative or other losses of indicator 128. This arrangement maximizes system integrity since fiducial rods 120 do not need to be refilled to compensate for evaporative or other losses. Further still, fiducial rods 120 are replaceable thereby allowing stereotactic head frame localizer 50 to be modified for use in other imaging modalities by replacing all fiducial rods 120 with ones suitable for the selected imaging modality (i.e. CT, MRI, or PET). In addition, some or all fiducial rods 120 may be replaced by fiducial rods 120 having different shapes and/or sizes for determining the orientation of stereotactic head frame localizer 50. Fiducial rods 120 may also be replaced so that stereotactic head frame localizer 50 includes fiducial rods 120 having fresh indicator 128 as may be done during routine or periodic maintenance.

Stereotactic head frame localizer 50 has a fixed relation to the patient that may be accomplished by direct clamping to the head, shown in phantom in FIG. 4A, using head ring 1′. Attached to head ring 1′ is stereotactic head frame localizer 50 such that when the selected scan modality is applied (i.e. CT, MRI, or PET), and a tomographic slice is imaged, then fiducial rod 120 intersections with the scan plane appear as localizer spots on the resulting image. Stereotactic head frame localizer 50 has a fixed relationship to the head ring 1′ and thus can be related to a set of coordinate axes (x, y, and z) and their origin 0 defined relative to the head ring 1′. The axial scheme of prior art involved the axial rods extending between points 14-18, 15-19, 16-20, 17-21, and the diagonal rods extending between points 17-18, 14-19, 16-19, 17-20. The diagonals may be oriented differently from what is shown in the specific example of FIG. 4A and accomplish the same effect. For example, the rods may go from points 14 to 21, 15 to 18, 15 to 20, or 16 to 21 or any combination of these pairs. They need not go exactly through the end points of the rods as shown, but may be offset, or displaced parallel, or at different angles. They need only be arranged so that when the scan plane intersects the localizer as shown in FIG. 2A, then from the image points of the intersection of the plane with the rods and the diagonals one can calculate the x, y, z position of each of the intersection points relative to the head ring 1′ fixed to the patient.

The embodiment in FIG. 4A includes fiducial rods 120 disposed in both horizontal and diagonal orientations, as previously discussed, that enable the determination of targets for other than nearly axial planar scan cuts. Fiducial rods 120 extending between points 17-14, 14-15, 15-16, 16-17 at the base of stereotactic head frame localizer 50, and fiducial rods 120 extending between points 18-21, 18-19, 19-20, 20-21 at the top of stereotactic head frame localizer 50 are oriented in a plane which is perpendicular to the axial rods (i.e. fiducial rods 120 extending between points 14-18, 15-19, 16-20, and 17-21) that is in the axial plane relative to the head ring 1′. Fiducial rod 120 of top side 100 that extends between points 18-20 is also in the axial plane (i.e. perpendicular to the axial direction).

The placement of fiducial rods 120 within stereotactic head frame localizer 50 are such that a set of nine reference marks will be visible in the basic orthogonal MR scan sets (axial, coronal, and sagittal). In these scans, the gel material in fiducial rods 120 is visible, and the marks show up as circular or elliptical solid dots around the perimeter of the head.

In CT scans (which are axial only), the glass casing of fiducial rods 120 can be differentiated from the rest of the frame structure, and the nine marks show up as circular or elliptical rings.

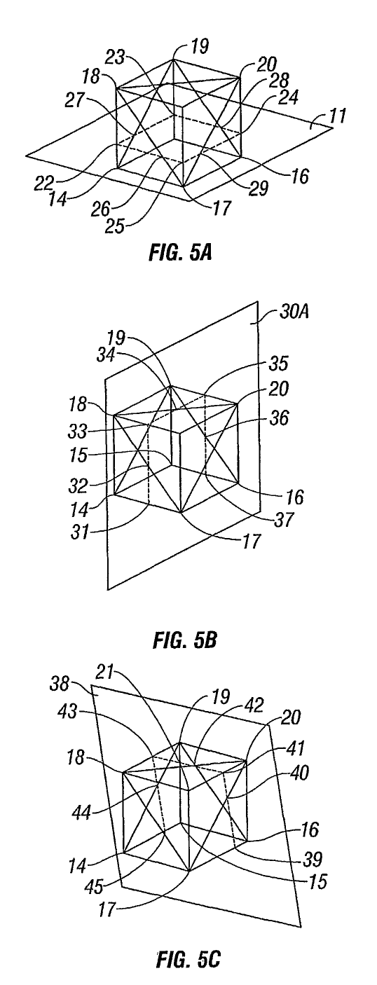

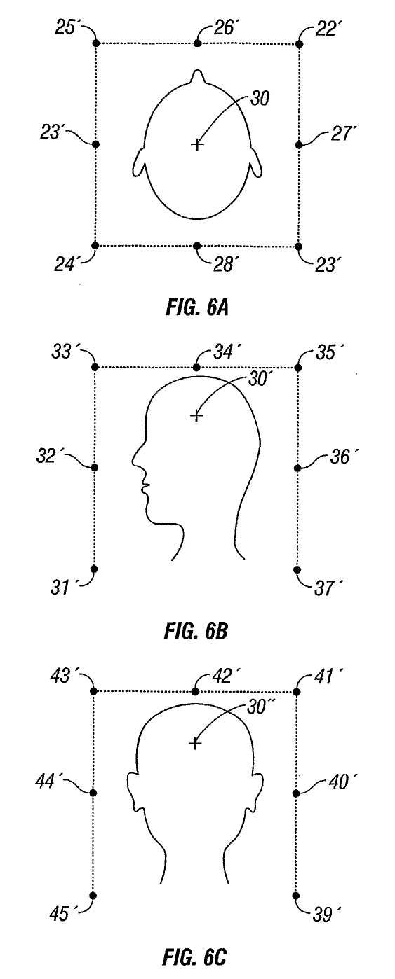

The orientation of fiducial rods 120 in stereotactic head frame localizer 50 permit sagittal and coronal slices or reconstructions to include localizer image spots from which target coordinates from the slice images can be determined, as illustrated in FIGS. 5A-C and 6A-C. First consider the axial plane scan cut of stereotactic head frame localizer 50 (FIG. 4A), shown in FIG. 5A. We will refer to planes which are essentially in the axial plane, but may be tilted somewhat from it as axial planes. The ideal axial plane we can consider as perpendicular to the Z-axis of the head ring 1′. Plane 11 intersects four axial fiducial rods 120 at points 22, 23, 24, 25, and their connecting diagonal fiducial rods 120 at points 26, 27, 28, and 29. Their images are shown as points on the axial scan cut image in FIG. 6A, designated as 22′, 23′, 24′, 25′ and 26′, 27′, 28′, 29′, respectively. A target 30 inside the body is seen on the image also. The proportional distance of 29′ from 25′ and 24′ for instance enable the x, y, and z coordinates of point 29 to be calculated relative to the reference head ring 1′. The same is true for diagonal points 26, 27, and 28. Thus, from any three of these coordinates, plane 11 can be calculated, and from the relative distances of target image 30 from the rest of the localizer image points in FIG. 6A, the x, y, and z coordinates of the real target in the body relative to head ring 1′ coordinate axis can also be calculated.

Now consider a nearly sagittal plane 30A as shown in FIG. 5B through the localizer and body. It intersects fiducial rods 120 at points 31, 33, 35, 37. The diagonal fiducial rods 120 are intersected at points 32, 34, 36. The image on the sagittal plane is shown in FIG. 6B as image points 31′, 32′, 33′, 34′, 35′, 36′, 37′, and the target image is 30′. From these image points, the target coordinates can be calculated relative to the head ring 1′ (FIG. 4A). By including fiducial rods 120 which are not parallel to the axial direction, image points 31′, 33′, 35′, and 37′ are obtainable. In addition, the inclusion of a diagonally oriented fiducial rod 120 in top side 100 allows for capturing image point 34′. These points are essential for calculating coordinates of points 32, 34, 36 and also for calculating plane 30A relative to head ring 1′. Using this acquired information, the coordinates of target 30′ relative to head ring 1′ can be calculated.

FIGS. 5C and 6C show the similar situation for a nearly coronal scan cut 38 which intersects fiducial rods 120 at points 39, 40, 41, 42, 43, 44, and 45, giving rise to coronal image points 39′, 40′, 41′, 42′, 43′, 44′, and 45′. An image target is depicted as image point 30″ (FIG. 6C). As discussed hereinabove, including fiducial rods 120 disposed at the base and top of stereotactic head frame localizer 50 and a diagonally oriented fiducial rod 120 in top side 100 enables these images to be seen and thus enable the target associated with image point 30″ to be determined in space relative to head ring 1′.

By adjusting or altering the nature, order, size, or orientation of one or more selected fiducial rods 120, the resulting image points may be used to determine which plane is being imaged. For example, in FIGS. 6A-C, image points 24′, 31′, and 45′ are made larger by including thicker fiducial rods 120 elements in stereotactic head frame localizer 50. This indexes the resulting image points so that the orientation of the slice is readily determinable for any of the three types of scan cuts (i.e. axial, sagittal, or coronal). Furthermore, by using different sectional shapes or sizes of fiducial rods 120, the sagittal versus coronal scan cuts or the parity of the image that is being viewed may be uniquely identifiable. Thus, a unique identification of the scanned plane and its orientation, for any plane in full 3-dimensions, is possible.

If head ring 1′ (FIG. 4A) is positioned so that axial scan cuts are parallel to it, then fewer localizer elements may be needed to define the plane of a slice and to determine target coordinates. Still, for coronal or sagittal scan cuts, the presence of at least one diagonally oriented fiducial rod 120 on top side 100, which is parallel to the axial plane (i.e. perpendicular to the axial direction), is essential to defining precisely the plane of a scan cut which is approximately, but not quite exactly, in either of the coronal or sagittal planes.

The present disclosure not only applies to imaging systems that explicitly derive the sagittal, coronal, or axial scan cuts, but also those imaging systems which reconstruct a series of axial scan cuts to generate a volumetric image, and then project out planar images subsequently in any arbitrary plane such as sagittal or coronal. Thus, if a series of axial scan cuts using stereotactic head frame localizer 50 (FIG. 4A) is taken, then fiducial rods 120 which are parallel to the axial plane will appear in certain axial scan cuts. When all axial scan cuts are reassembled in a computer, mathematical projections in other planes will intersect fiducial rods 120, including diagonally oriented fiducial rods 120, and show them as point spots. Other imaging techniques using time-swept scanning (as in certain MRI techniques) will similarly produce spot reconstructed image intersections with stereotactic head frame localizer's 50 fiducial rods 120.

While the above description contains many specifics, these specifics should not be construed as limitations on the scope of the present disclosure, but merely as exemplifications of preferred embodiments thereof. Although the present disclosure is directed towards a stereotactic head frame localizer and associated fiducial rods, alternate embodiments of the present disclosure are contemplated for use in scanning other regions of a body. Those skilled in the art will envision many other possible variations that are within the scope and spirit of the present disclosure.

What is claimed is:

1. A surgical apparatus usable with a support structure, the support structure capable of being releasably attached to a portion of a patient's anatomy, the surgical apparatus comprising:
a plurality of connected sides wherein at least one side is releasably attached to the surgical apparatus;
a plurality of rods wherein each side of the surgical apparatus includes at least one rod and the plurality of rods includes at least three diagonally oriented rods;
an indicator disposed in each of the rods, the indicator being substantially opaque to a selected imaging modality.

2. The surgical apparatus of claim 1, wherein each side of the surgical apparatus has no more than one diagonal rod disposed therein.

3. The surgical apparatus of claim 1, wherein the plurality of sides forms a geometric shape and defines at least one opening.

4. The surgical apparatus of claim 3, wherein the opening is configured and dimensioned to receive a head of the patient.

5. The surgical apparatus of claim 3, wherein the surgical apparatus further includes at least one side opposite said opening.

6. The surgical apparatus of claim 3, wherein each side includes at least one diagonally oriented rod and at least one other rod.

7. The surgical apparatus of claim 6, wherein the at least one other rod is oriented in a vertical [or a horizontal] direction.

8. The surgical apparatus of claim 6, wherein the at least one other rod is oriented in a horizontal direction.

9. The surgical apparatus of claim 1, wherein each rod has a closed end and an open end defining a channel therein for receiving a quantity of the indicator therein.

10. The surgical apparatus of claim 8, wherein the open end of each rod is sealed thereby containing quantity of the indicator in each rod.

11. The surgical apparatus of claim 9, wherein each rod is sealed using a selected adhesive.

12. A surgical apparatus usable with a support structure, the support structure capable of being releasably attached to a portion of a patient's anatomy, the surgical apparatus comprising:
a plurality of connected sides including an anterior side, a left side, a right side, a top side and a posterior side, wherein at least one side is releasably attached to the surgical apparatus;
a plurality of rods wherein each side of the surgical apparatus includes at least one rod and the plurality of rods includes at least three diagonally oriented rods; and
an indicator disposed in each of the rods, the indicator being substantially opaque to a selected imaging modality.

13. The surgical apparatus as recited in claim 12, wherein the posterior side is removable.

14. The surgical apparatus as recited in claim 12, wherein each of the rods is permanently sealed against modification by the user.

15. The surgical apparatus as recited in claim 12, wherein at least one rod of the plurality of rods is thicker than the remaining rods.

16. A fiducial rod for use with a stereotactic head localizer comprising:
an elongate tubular structure having a core defining a space and having first and second opposed ends; and
an indicator at least partially filing the core, wherein the first and second ends of the elongate tubular structure are permanently sealed against opening by the user.

17. The fiducial rod as recited in claim 16, wherein the elongate tubular structure is formed of glass.

18. The fiducial rod as recited in claim 16, wherein the first and second ends are sealed by caps.

19. The fiducial rod as recited in claim 16, wherein the first and second ends are sealed by plugs.
